(19) United States
(12) Patent Application Publication
(10) Pub. No.: US 2008/0091207 A1
(43) Pub. Date: Apr. 17, 2008

(54) BONE TREATMENT SYSTEMS AND METHODS

(76) Inventors: Csaba Truckai, Saratoga, CA (US); Robert Luzzi, Pleasanton, CA (US); John H. Shadduck, Tiburon, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/872,649

(22) Filed: Oct. 15, 2007

Related U.S. Application Data
(60) Provisional application No. 60/851,682, filed on Oct. 13, 2006.

(51) Int. Cl. A61B 17/00; A61B 17/58

(52) U.S. Cl. 606/79; 606/103; 606/62; 606/92

(57) ABSTRACT

Apparatuses, methods, and kits for treating bone (e.g., vertebral compression fractures) includes a shaft with a working end that can be bent into an arc-shaped working end. The shaft can be introduced into a bone (e.g., introduced through a sleeve into cancellous bone) and carries a cutting element that can be actuated across the arc-shaped working end to cut a plane in cancellous bone, which can optionally be filled with a bone fill material (e.g., bone cement).

### CROSS-REFERENCE TO RELATED APPLICATIONS

This application claims the benefit of U.S. Provisional Patent Application No. 60/851,682 filed Oct. 13, 2006, the entire contents of which are incorporated herein by reference and should be considered a part of this specification. This application is related to the following U.S. patent application Ser. No. 11/469,764 filed Sep. 1, 2006 titled Methods for Sensing Retrograde Flows of Bone Fill Material, Ser. No. 11/165,652 filed Jun. 24, 2005 titled Bone Treatment Systems and Methods; Ser. No. 60/726,152 (Docket No. S-7700-310) filed Oct. 13, 2005 titled Bone Treatment Systems and Methods; and Ser. No. 11/209,035 (Docket No. S-7700-280) filed Aug. 22, 2005, titled Bone Treatment Systems and Methods. The entire contents of all of the above applications are hereby incorporated by reference and should be considered a part of this specification.

### BACKGROUND OF THE INVENTION

1. Field of the Invention

The present invention relates in certain embodiments to systems for treating vertebral compression fractures, and more particularly to a device and method for cutting a plane in a cancellous bone portion of a bone.

2. Description of the Related Art

Osteoporotic fractures are prevalent in the elderly, with an annual estimate of 1.5 million fractures in the United States alone. These include 750,000 vertebral compression fractures (VCFs) and 250,000 hip fractures. The annual cost of osteoporotic fractures in the United States has been estimated at $13.8 billion. The prevalence of VCFs in women age 50 and older has been estimated at 26%. The prevalence increases with age, reaching 40% among 80-year-old women. Medical advances aimed at slowing or arresting bone loss from aging have not provided solutions to this problem. Further, the population affected will grow steadily as life expectancy increases. Osteoporosis affects the entire skeleton but most commonly causes fractures in the spine and hip. Spinal or vertebral fractures also cause other serious side effects, with patients suffering from loss of height, deformity and persistent pain which can significantly impair mobility and quality of life. Fracture pain usually lasts 4 to 6 weeks, with intense pain at the fracture site. Chronic pain often occurs when one vertebral level is greatly collapsed or multiple levels are collapsed.

Postmenopausal women are predisposed to fractures, such as in the vertebrae, due to a decrease in bone mineral density that accompanies postmenopausal osteoporosis. Osteoporosis is a pathologic state that literally means “porous bones”. Skeletal bones are made up of a thick cortical shell and a strong inner meshwork, or cancellous bone, of collagen, calcium salts and other minerals. Cancellous bone is similar to a honeycomb, with blood vessels and bone marrow in the spaces. Osteoporosis describes a condition of decreased bone mass that leads to fragile bones which are at an increased risk for fractures. In an osteoporotic bone, the sponge-like cancellous bone has pores or voids that increase in dimension making the bone very fragile. In young, healthy bone tissue, bone breakdown occurs continually as the result of osteoclast activity, but the breakdown is balanced by new bone formation by osteoblasts. In an elderly patient, bone resorption can surpass bone formation thus resulting in deterioration of bone density. Osteoporosis occurs largely without symptoms until a fracture occurs.

Vertebroplasty and kyphoplasty are recently developed techniques for treating vertebral compression fractures. Percutaneous vertebroplasty was first reported by a French group in 1987 for the treatment of painful hemangiomas. In the 1990's, percutaneous vertebroplasty was extended to indications including osteoporotic vertebral compression fractures, traumatic compression fractures, and painful vertebral metastasis. Vertebroplasty is the percutaneous injection of PMMA (polymethylmethacrylate) into a fractured vertebral body via a trocar and cannula. The targeted vertebrae are identified under fluoroscopy. A needle is introduced into the vertebrae body under fluoroscopic control, to allow direct visualization. A bilateral transpedicular (through the pedicle of the vertebrae) approach is typical but the procedure can be done unilaterally. The bilateral transpedicular approach allows for more uniform PMMA infill of the vertebra.

In a bilateral approach, approximately 1 to 4 ml of PMMA is used on each side of the vertebra. Since the PMMA needs to be forced into the cancellous bone, the techniques require high pressures and fairly low viscosity cement. Since the cortical bone of the targeted vertebra may have a recent fracture, there is the potential of PMMA leakage. The PMMA cement contains radiopaque materials so that when injected under live fluoroscopy, cement localization and leakage can be observed. The visualization of PMMA injection and extravasation are critical to the technique—and the physician terminates PMMA injection when leakage is evident. The cement is injected using syringes to allow the physician manual control of injection pressure.

Kyphoplasty is a modification of percutaneous vertebroplasty. Kyphoplasty involves a preliminary step consisting of the percutaneous placement of an inflatable balloon tamp in the vertebral body. Inflation of the balloon creates a cavity in the bone prior to cement injection. The proponents of percutaneous kyphoplasty have suggested that high pressure balloon-tamp inflation can at least partially restore vertebral body height. In kyphoplasty, some physicians state that PMMA can be injected at a lower pressure into the collapsed vertebra since a cavity exists, when compared to conventional vertebroplasty.

The principal indications for any form of vertebroplasty are osteoporotic vertebral collapse with debilitating pain. Radiography and computed tomography must be performed in the days preceding treatment to determine the extent of vertebral collapse, the presence of epidural or foraminal stenosis caused by bone fragment retropulsion, the presence of cortical destruction or fracture and the visibility and degree of involvement of the pedicles.

Leakage of PMMA during vertebroplasty can result in very serious complications including compression of adjacent structures that necessitate emergency decompressive surgery. See “Anatomical and Pathological Considerations in Percutaneous Vertebroplasty and Kyphoplasty: A Reappraisal of the Vertebral Venous System”, Groen, R. et al, Spine Vol. 29, No. 13, pp 1465-1471 2004. Leakage or extravasation of PMMA is a critical issue and can be divided into paravertebral leakage, venous infiltration, epidural leakage and intradiscal leakage. The exothermic reaction of PMMA carries potential catastrophic consequences if thermal damage were to extend to the dural sac, cord, and nerve roots. Surgical evacuation of leaked cement in the spinal canal has been reported. It has been found that leakage of PMMA is related to various clinical factors such as the vertebral compression pattern, and the extent of the cortical fracture, bone mineral density, the interval from injury to operation, the amount of PMMA injected and the location of the injector tip. In one recent study, close to 50% of vertebroplasty cases resulted in leakage of PMMA from the vertebral bodies. See Hyun-Woo Do et al, “The Analysis of Polymethylmethacrylate Leakage after Vertebroplasty for Vertebral Body Compression Fractures”, Jour. of Korean Neurosurg. Soc. Vol. 35, No. 5 (May 2004) pp. 478-82, (http://www.jkns.or.kr/htm/abstract.asp?no=0042004086).

Another recent study was directed to the incidence of new VCFs adjacent to the vertebral bodies that were initially treated. Vertebroplasty patients often return with new pain caused by a new vertebral body fracture. Leakage of cement into an adjacent disc space during vertebroplasty increases the risk of a new fracture of adjacent vertebral bodies. See Am. J. Neuroradiol. 2004 February; 25(2):175-80. The study found that 58% of vertebral bodies adjacent to a disc with cement leakage fractured during the follow-up period compared with 12% of vertebral bodies adjacent to a disc without cement leakage.

Another life-threatening complication of vertebroplasty is pulmonary embolism. See Bernhard, J. et al, “Asymptomatic diffuse pulmonary embolism caused by acrylic cement: an unusual complication of percutaneous vertebroplasty”, Ann. Rheum. Dis. 2003; 62:85-86. The vapors from PMMA preparation and injection also are cause for concern. See Kirby, B, et al., “Acute bronchospasm due to exposure to polymethylmethacrylate vapors during percutaneous vertebroplasty”, Am. J. Roentgenol. 2003; 180:543-544.

In both higher pressure cement injection (vertebroplasty) and balloon-tamped cementing procedures (kyphoplasty), the methods do not provide for well controlled augmentation of vertebral body height. The direct injection of bone cement simply follows the path of least resistance within the fractured bone. The expansion of a balloon also applies to compacting forces along lines of least resistance in the collapsed cancellous bone. Thus, the reduction of a vertebral compression fracture is not optimized or controlled in high pressure balloons as forces of balloon expansion occur in multiple directions.

In a kyphoplasty procedure, the physician often uses very high pressures (e.g., up to 200 or 300 psi) to inflate the balloon which crushes and compacts cancellous bone. Expansion of the balloon under high pressures close to cortical bone can fracture the cortical bone, typically the endplates, which can cause regional damage to the cortical bone with the risk of cortical bone necrosis. Such cortical bone damage is highly undesirable as the endplate and adjacent structures provide nutrients for the disc.

Kyphoplasty also does not provide a distraction mechanism capable of 100% vertebral height restoration. Further, the kyphoplasty balloons under very high pressure typically apply forces to vertebral endplates within a central region of the cortical bone that may be weak, rather than distributing forces over the endplate.

There is a need for improved devices, systems and methods for use in the treatment of vertebral compression fractures.

### SUMMARY OF THE INVENTION

In accordance with one embodiment, a method for treating bone is provided. The method comprises providing an elongated shaft capable of linear and curved shapes about a flex axis, and a shaft-associated cutting element that is adapted to assume a first configuration co-linear the shaft and flex axis and adapted to assume a second configuration that is not co-linear with the shaft and flex axis when the shaft is curved. The method also comprises positioning the shaft in cancellous bone in a curved shape and actuating the cutting element from the first configuration to the second configuration thereby creating a cut plane in the cancellous bone.

In accordance with another embodiment, a method for treating a vertebra is provided. The method comprises providing a shape memory shaft with a repose arc-shaped working end with a wire-like cutting element that is extendable away from the working end in a plane across the arc-shape. The method also comprises introducing the working end into cancellous bone in a vertebra, extending the cutting element away from the working end to thereby cut a plane in the cancellous bone, and introducing a bone cement flow into the plane in the cancellous bone.

In accordance with another embodiment, a method of treating a vertebra is provided. The method comprises providing a shaft having an arc-configurable working end with a wire-like cutting element that is extendable away from the working end in a plane across the arc-shape, introducing the working end into cancellous bone in a vertebra, causing the working end to extend at least about 90° in an arc configuration in the cancellous bone, and extending a cutting element from the working end across the arc configuration to thereby cut bone.

In accordance with still another embodiment, a device for treating bone is provided. The device comprises an elongated shaft capable of linear and curved shapes relative to an axis. The device also comprises an elongated cutting element carried by the shaft, the cutting element adapted to assume a first configuration co-linear the shaft and adapted to assume a second configuration that is not co-linear with the shaft when the shaft is curved.

In accordance with still another embodiment, a device for treating bone is provided. The device comprises an elongated shaft adapted for insertion through a cortical bone portion of a bone and into a cancellous bone portion of the bone, the elongated shaft having a linear shape extending along a longitudinal axis, at least a portion of the shaft being movable into a curved configuration within cancellous bone. The device also comprises a cutting element attached to said movable portion of the shaft, the cutting element moveable between a first configuration co-linear with the shaft and a second configuration away from the shaft with the movable portion of the shaft in the curved configuration to cut a plane in cancellous bone.

In accordance with still another embodiment, a device for treating bone is provided. The device comprises an elongated shaft adapted for insertion through a cortical bone portion of a bone and into a cancellous bone portion of the bone, the elongated shaft having a linear shape along a longitudinal axis, at least a portion of the shaft movable into a curved configuration within cancellous bone. The device also comprises a means for cutting a plane in cancellous bone, said means being attached to at least a portion of the elongated shaft.

In accordance with yet another embodiment, a method of treating bone is provided. The method comprises creating a path into a cancellous bone portion in a bone, inserting an elongated shaft along a longitudinal axis through said path into cancellous bone, the shaft having a cutting element attached to a working end of the shaft, moving the working end of the shaft into a curved configuration, and cutting a plane in cancellous bone with the cutting element.

In accordance with yet another embodiment, a kit for treating bone is provided. The kit comprises an injector configured for introduction into a bone, the injector configured to deliver bone cement through a channel thereof into the bone. The kit also comprises a cutting tool comprising an elongated shaft adapted for insertion through a cortical bone portion of the bone and into a cancellous bone portion of the bone, and a cutting element attached to said portion of the shaft, the cutting element moveable between a first configuration co-linear with the shaft and a second configuration not co-linear with the shaft to cut a plane in cancellous bone

These and other objects of the present embodiments of the invention will become readily apparent upon further review of the following drawings and specification.

### BRIEF DESCRIPTION OF THE DRAWINGS

In order to better understand the invention and to see how it may be carried out in practice, some preferred embodiments are next described, by way of non-limiting examples only, with reference to the accompanying drawings, in which like reference characters denote corresponding features consistent throughout similar embodiments in the attached drawings.

FIG. 1 is a schematic perspective view of one embodiment of a bone cement injection system.

FIG. 2 is another schematic view of the bone cement injector of FIG. 1.

FIG. 3A is a schematic cross-sectional view of a vertebra showing a step in one embodiment of a bone cement injection method.

FIG. 3B is a schematic cross-sectional view of the vertebra of FIG. 3A showing another step in a bone cement injection method.

FIG. 3C is a schematic cross-sectional view similar to FIGS. 3A-3B showing another step in a bone cement injection method.

FIG. 4 is a schematic cut-away view of another embodiment of a bone cement injector similar to that of FIGS. 1-2.

FIG. 5 is a schematic cross-sectional view of a distal portion of the bone cement injector of FIGS. 1-2.

FIG. 6 is a schematic view of one embodiment of a bone pathway-forming shaft adapted to deflect and provide a curved path in cancellous bone, the shaft carrying a cutting element.

FIG. 7 is a schematic cross-sectional view of the shaft of FIG. 1 with the cutting element carried in a channel.

FIG. 8A is a schematic view of a step of one embodiment of a method of accessing cancellous bone, showing the advancement of the shaft of FIG. 6 through cortical bone of the pedicle.

FIG. 8B is a schematic view of another step of a method of accessing cancellous bone, showing the advancement of the shaft of FIG. 6 in cancellous bone.

FIG. 8C is a schematic view of another step of a method for accessing cancellous bone, showing actuation of the cutting element to cut a plane in the cancellous bone.

### DETAILED DESCRIPTION OF THE PREFERRED EMBODIMENTS

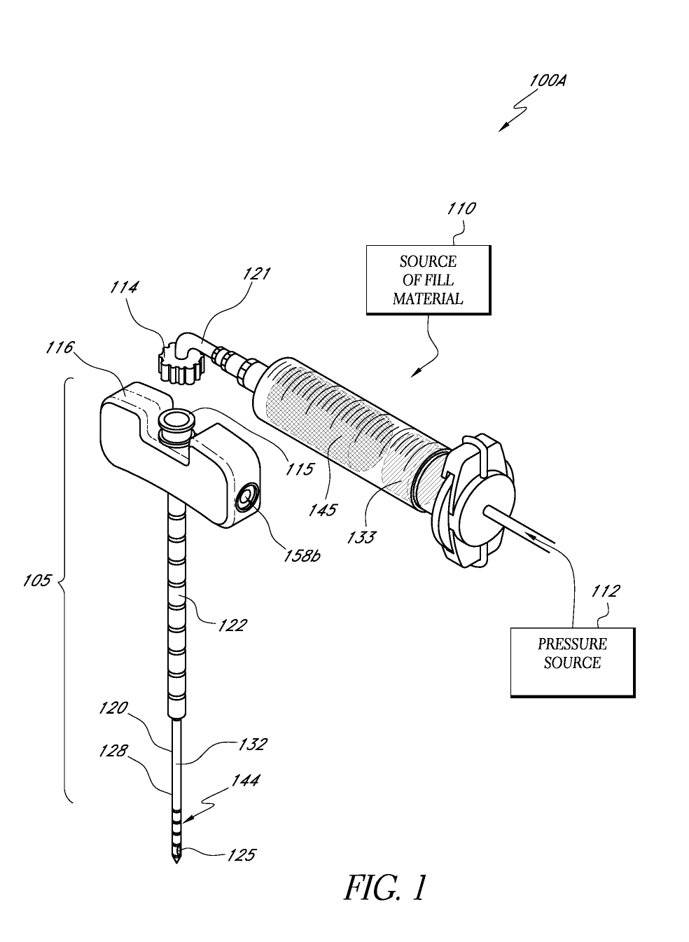

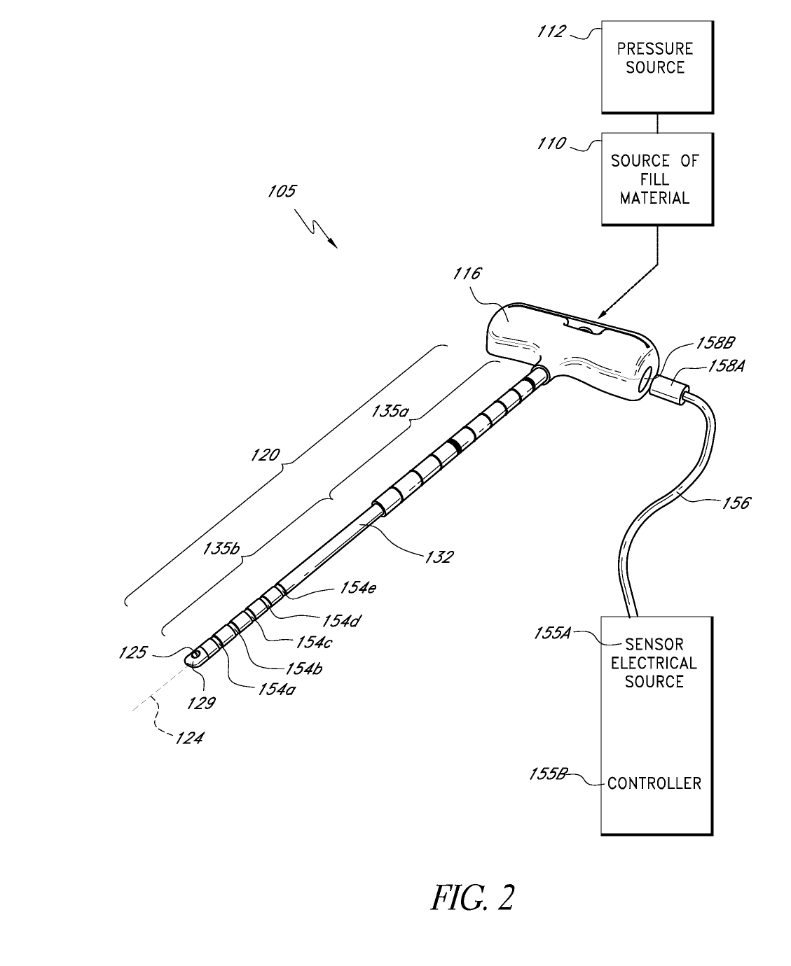

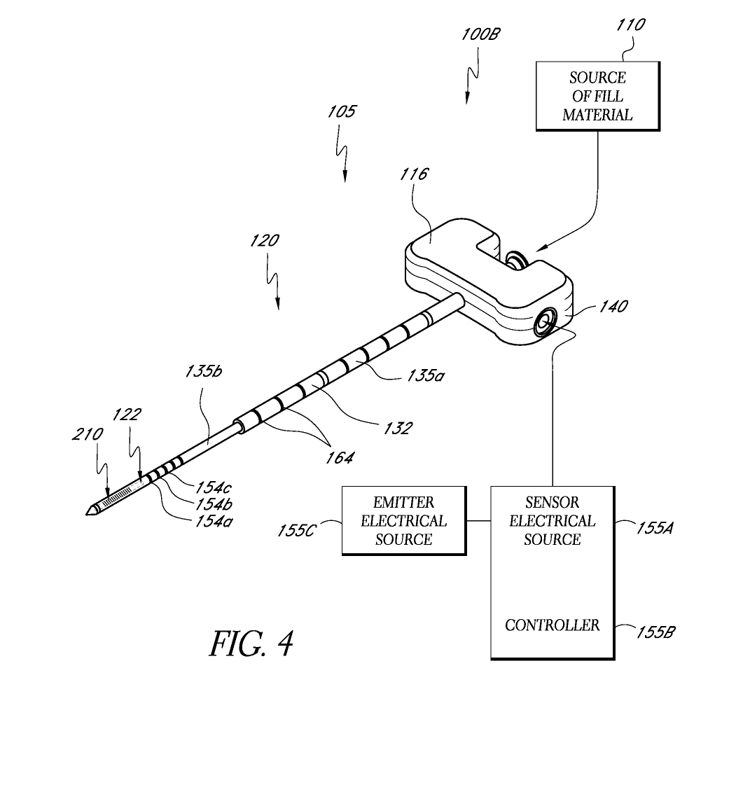

FIGS. 1-2 show one embodiment of a bone fill introducer or injector system 100A for treatment of the spine in a vertebroplasty procedure. The system 100A can include a bone cement injector 105 coupled to a bone fill material source 110, wherein the injection of the fill material is carried out by a pressure mechanism or source 112 operatively coupled to source 110 of the bone fill material. In one embodiment as in FIG. 1, the pressure source 112 is a hydraulic actuator that is computer controlled. However, in another embodiment, a manually operated syringe loaded with bone fill material, or any other pressurized source of fill material, can be used. The source 110 of fill material includes a coupling or fitting 114 for sealable locking to a cooperating fitting 115 at a proximal end or handle 116 (also see FIG. 4) of the bone cement injector 105 that has an elongated introducer sleeve indicated at 120. In one embodiment, a syringe-type source 110 can be coupled directly to fitting 115 with a flexible, rigid or bendable (deformable) hydraulic tube 121 extending to pressure source 112. The fill material then can flow through handle 116 to communicate with a passageway 122 in introducer sleeve 120.

As background, a vertebroplasty procedure using any of the embodiments disclosed herein would include inserting the introducer of FIG. 1 through a pedicle of a vertebra for accessing the osteoporotic cancellous bone. The initial aspects of the procedure are similar to a conventional percutaneous vertebroplasty wherein the patient is placed in a prone position on an operating table. The patient is typically under conscious sedation, although general anesthesia is an alternative. The physician injects a local anesthetic (e.g., 1% Lidocaine) into the region overlying the targeted pedicle or pedicles as well as the periosteum of the pedicle(s). Thereafter, the physician uses a scalpel to make a 1 to 5 mm skin incision over each targeted pedicle. Thereafter, the introducer is advanced through the pedicle into the anterior region of the vertebral body, which typically is the region of greatest compression and fracture. The physician confirms the introducer path posterior to the pedicle, through the pedicle and within the vertebral body by anteroposterior and lateral X-Ray projection fluoroscopic views. The introduction of infill material as described below can be imaged several times, or continuously, during the treatment depending on the imaging method.

### DEFINITIONS

“Bone fill, fill material, or infill material or composition” includes its ordinary meaning and is defined as any material for infilling a bone that includes an in-situ hardenable material or that can be infused with a hardenable material. The fill material also can include other “fillers” such as filaments, microspheres, powders, granular elements, flakes, chips, tubules and the like, autograft or allograft materials, as well as other chemicals, pharmacological agents or other bioactive agents.

“Flowable material” includes its ordinary meaning and is defined as a material continuum that is unable to withstand a static shear stress and responds with an irrecoverable flow (a fluid)—unlike an elastic material or elastomer that responds to shear stress with a recoverable deformation. Flowable material includes fill material or composites that include a fluid (first) component and an elastic or inelastic material (second) component that responds to stress with a flow, no matter the proportions of the first and second component, and wherein the above shear test does not apply to the second component alone.

“Substantially” or “substantial” mean largely but not entirely. For example, substantially may mean about 10% to about 99.999%, about 25% to about 99.999% or about 50% to about 99.999%.

“Osteoplasty” includes its ordinary meaning and means any procedure wherein fill material is delivered into the interior of a bone.

“Vertebroplasty” includes its ordinary meaning and means any procedure wherein fill material is delivered into the interior of a vertebra.

In FIGS. 1-5, it can be seen that elongated introducer sleeve 120 of bone cement injector 105 includes an interior channel 122 extending about axis 124 wherein the channel 122 terminates in a distal open outlet 125. The outlet 125 can be a single opening or a plurality of openings about the radially outward surface 128 of sleeve 120 or an opening at the distal tip 129 of the sleeve. The distal tip 129 can be blunt or sharp. In one embodiment, a core portion 130 of sleeve 120 is an electrically conductive metal sleeve, such as a stainless steel hypo tube. The core sleeve portion 130 has both an exterior insulative coating 132 and an interior insulative coating that will be described in greater detail below.

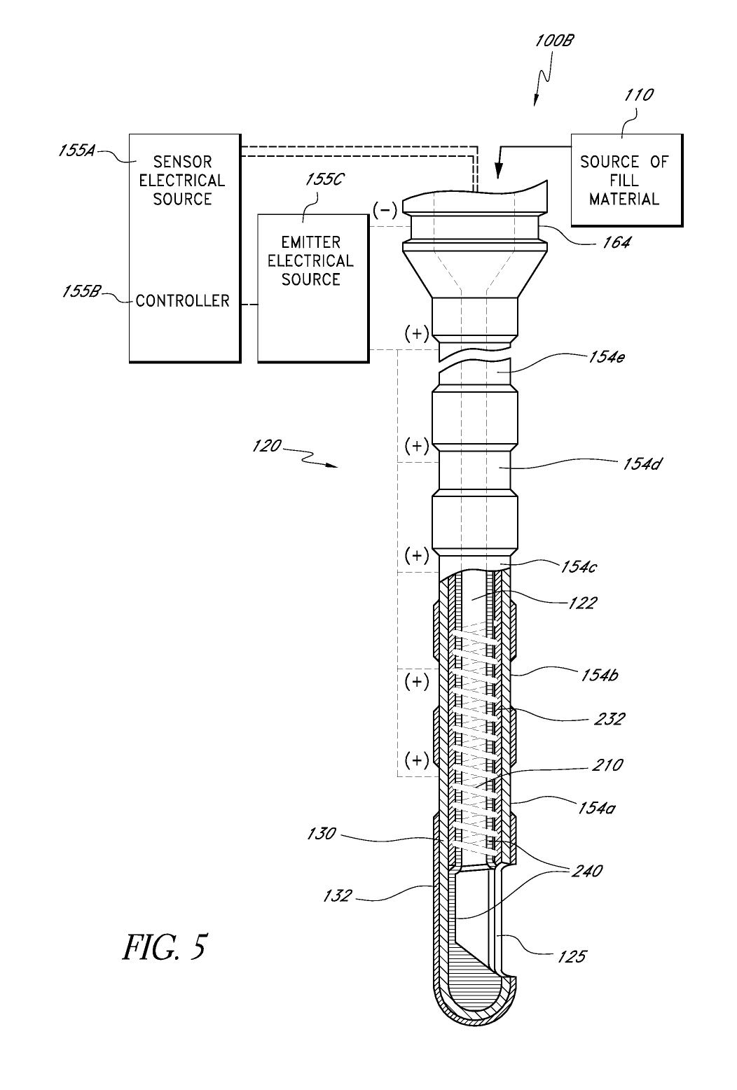

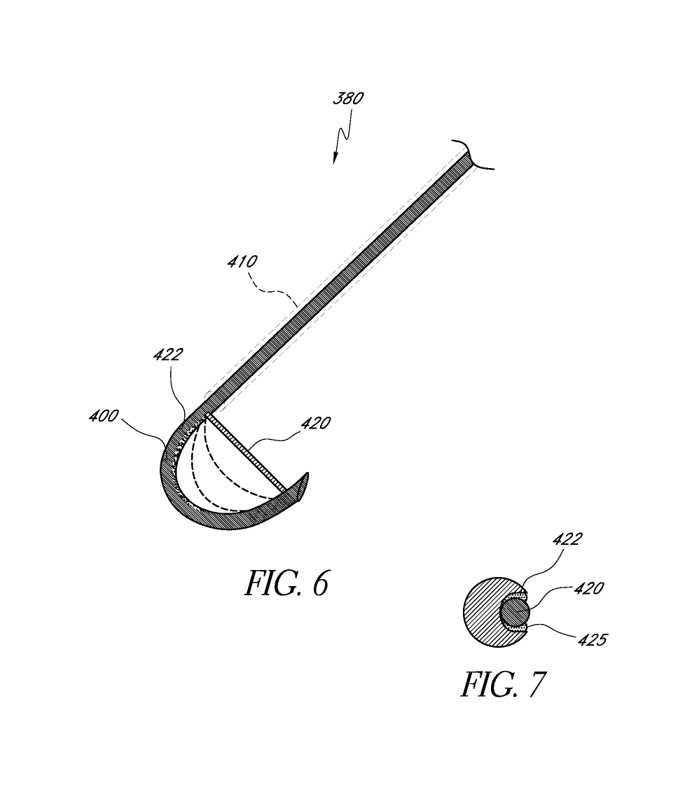

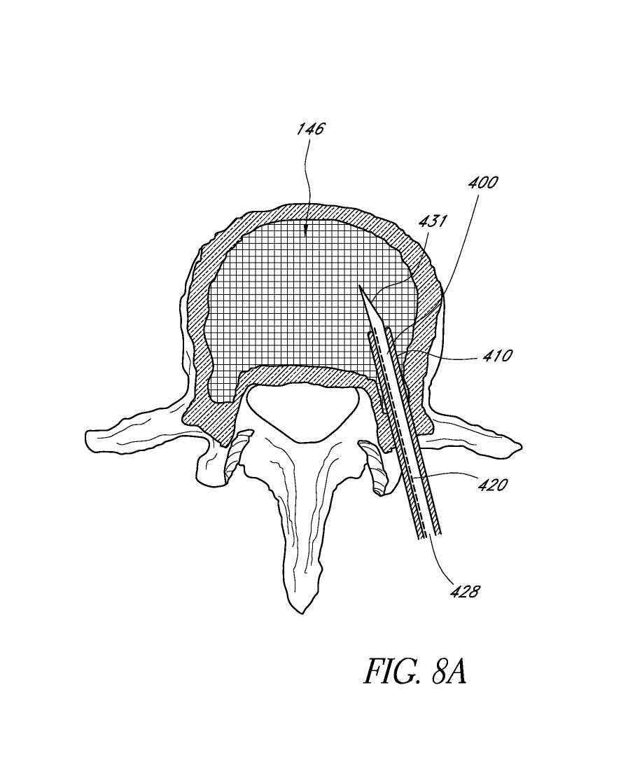

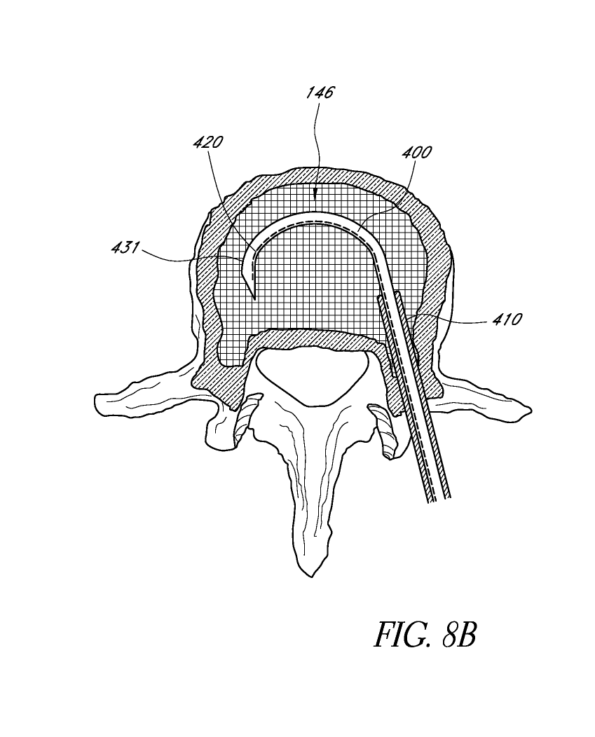

In one embodiment as shown in FIGS. 1-2, the bone fill system 100A has a container of fill material source 110 (also see FIGS. 4-5) that is pressurized by a hydraulic source acting on a floating piston 133 (phantom view) in the syringe-like source 110 that carries the fill material. In FIGS. 1-2, it can be seen that introducer sleeve 120 has a proximal portion 135a that is larger in cross-section than distal portion 135b (also see FIG. 4) with corresponding larger and smaller interior channel portions therein. This allows for lesser injection pressures since the cement flow may travel less distance through the smaller diameter distal portion of the introducer sleeve. In one embodiment, the distal portion 135b of the introducer can have a cross section ranging between about 2 mm and 4 mm with a length ranging between about 40 mm and 60 mm. The proximal portion 135a of introducer sleeve 120 can have a cross section ranging between about 5 mm and 15 mm, or between about 6 mm and 12 mm.

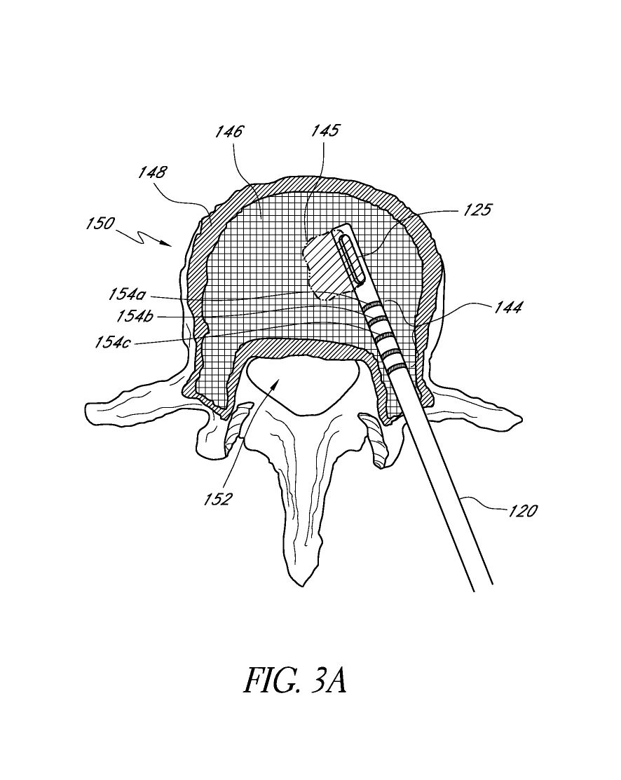

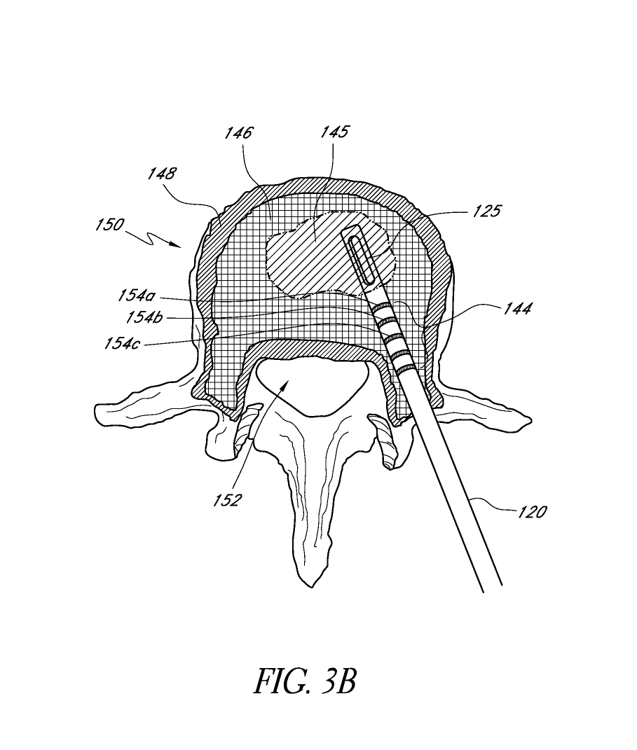

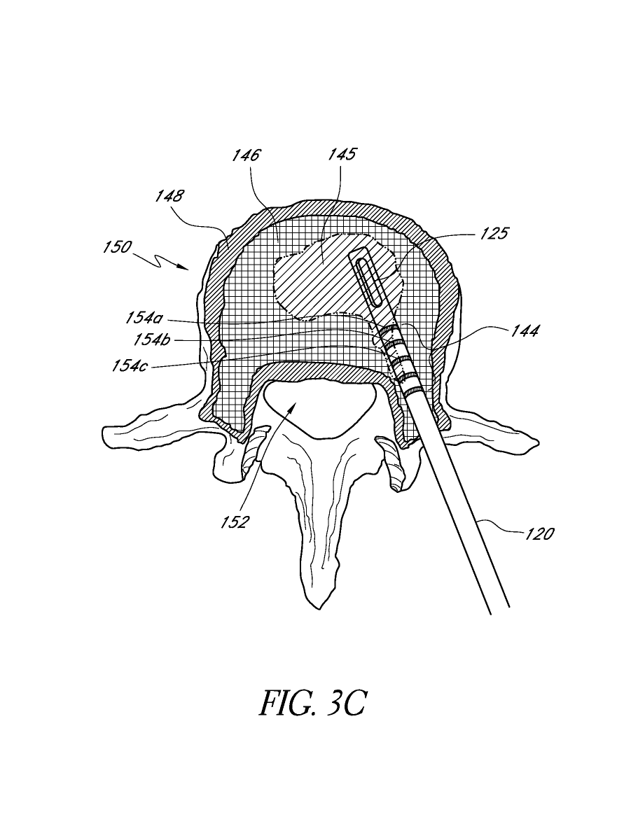

As can be seen in FIGS. 1-2, the exterior surface of introducer sleeve 120 can have a sensor system 144 that can sense the flow or movement of a fill material or cement 145 (also see FIGS. 3A-3C) proximate to the sensor system 144. The introducer sleeve 120 with such a sensor system 144 is particularly useful in monitoring and inhibiting extravasation of fill material 145 in a vertebroplasty procedure.

In one embodiment and method of use, referring to FIGS. 3A-3C, the introducer sleeve 120 can be used in a conventional vertebroplasty procedure with a single pedicular access or a bi-pedicular access. The fill material 145 can be a bone cement, such as PMMA, that is injected into cancellous bone 146 which is within the interior of the cortical bone surface 148 of a vertebra 150.

In FIGS. 3A-3B, it can be seen that a progressive flow of cement 145 is provided from outlet 125 of introducer sleeve 120 into the interior of the vertebra 150. FIG. 3A illustrates an initial flow volume, with FIG. 3B illustrating an increased flow volume of cement 145. FIG. 3C depicts a situation that is known to occur when bone is fractured along the entry path of introducer 120, wherein the cement 145 under high injection pressures finds the path of least resistance to be at least partly in a retrograde direction along the surface of introducer 120. The retrograde flow of cement as in FIG. 3C, if allowed to continue, could lead to cement extravasation into the spinal canal 152, which is undesirable. As can be understood from FIG. 3C, the sensor system 144 can be actuated when cement 145 comes proximate to, or into contact with, the sensor system. In one embodiment as shown in FIGS. 2-3C, the sensor system comprises a plurality of spaced apart exposed electrodes or electrode portions (e.g., electrodes 154a, 154b, 154c, etc.) coupled to a sensor electrical source 155A via a cable 156 and plug 158a connected to an electrical connector 158b (also see FIG. 1) in the proximal handle end of the introducer, wherein the electrical source 155A carries a low voltage direct current or Rf current between the opposing potentials of spaced apart electrodes. In one embodiment, the voltage can be from about 0.1 volt to 500 volts, or from about 1 volt to 5 volts, and can create a current path through the tissue between a pair of electrodes. The current can be continuous, intermittent and/or multiplexed between different electrode pairs or groups of electrodes. The arrangement of electrodes can be spaced apart ring-type electrodes and axially spaced apart as shown in FIGS. 1 and 2, or the electrodes can be discrete elements, helically spaced electrodes, or the electrodes can be miniaturized electrodes as in thermocouples, MEMS devices or any combination thereof. The number of sensors or electrodes can range from about 1 to 100 and can be adapted to cooperate with a ground pad or other surface portion of sleeve 120. In one embodiment, the electrodes can include a PTCR or NTCR material (positive temperature coefficient of resistance or negative temperature coefficient of resistance) and can function as thermistors to allow measurement of temperature, as well as functioning as a sensor. The sensor system 144 can include a controller 155B (FIG. 2) that measures at least one selected parameter of the current flow to determine a change in a parameter (e.g., impedance). When the non-conductive bone cement 145 contacts one or more electrodes of the sensor system 144, the controller 155B preferably identifies a change in the selected electrical parameter and can generate a signal to the operator. The scope of the invention includes, but is not limited to, sensor systems capable of sensing a change in electrical properties, reflectance, fluorescence, magnetic properties, chemical properties, mechanical properties or a combination thereof.

Now referring to FIGS. 4 and 5, an alternative injector system 100B includes bone cement injector 105 that is similar to the injector of FIGS. 1-2, but with a different embodiment of a sensor system together, and including an additional electrical energy delivery system for applying energy to fill material for altering its viscosity. In the illustrated embodiment, the ring electrode portions (e.g. electrodes 154a, 154b, 154c, etc. in phantom view) are exposed portions of a metal core portion 130 of sleeve 120 (see FIG. 5) that are coupled via a lead 140 to an electrical source 155A. The electrode portions 154a, 154b, 154c, etc. are indicated as having a first polarity (+) that cooperates with one or more second polarity (−) return electrodes 164 in a more proximal portion of the sleeve coupled by lead 140 to sensor electrical source 155A. Current can flow through the multiple electrode portions 154a, 154b, 154c, etc. and then through engaged tissue to the return electrodes 164, wherein the current flow will signal certain impedance parameters before and during an initial injection of cement 145, as shown in FIGS. 3A-3B. When there is a retrograde flow of cement 145, as in FIG. 3C, that covers one or more electrode portions 154a, 154b, 154c, etc., then the electrical parameter (e.g., impedance) changes to thus signal the operator that such a retrograde flow has contacted or covered an electrode portion 154a, 154b or 154c, etc. The change in parameter can be a rate of change in impedance, a change in impedance compared to a data library, etc. which will signal the operator of such a flow. The controller 155B also can automatically terminate the activation of pressure source 112 (see FIG. 1-2) upon receipt of said signal.

In the system of FIGS. 4 and 5, the bone fill injection system further includes a thermal energy emitter within a distal portion of interior channel 122 of the introducer 120 for heating a flow of bone cement from an open termination 125 in the introducer 120. In one embodiment, the thermal energy emitter is a resistive heating element 210 that can elevate the temperature of cement 145 (also see FIG. 1) to at least 50° C., at least 60° C., at least 70° C. or at least 80° C. In the illustrated embodiment, the resistive element 210 is coupled to emitter electrical source 155C as depicted in FIGS. 4 and 5 together with a controller 155B that can control cement inflow parameters such as variable flow rates, constant flow rates and/or pulsed flows in combination with controlled energy delivery. The thermal energy delivery is adapted to accelerate polymerization and increase the viscosity of a PMMA or similar bone cement as disclosed in the co-pending U.S. patent applications listed below. In another embodiment, the thermal energy emitter also can be an Rf emitter adapted for ohmically heating a bone cement that carries electrically conductive compositions as disclosed in the below co-pending U.S. patent application Ser. No. 11/165,652 filed Jun. 24, 2005; Ser. No. 11/165,651 filed Jun. 24, 2005; Ser. No. 11/208,448 filed Aug. 20, 2005; and Ser. No. 11/209,035 filed Aug. 22, 2005. In another embodiment, the thermal energy emitter can deliver thermal energy to bone cement and can be selected from the group consisting of a resistively heated emitter, a light energy emitter, an inductive heating emitter, an ultrasound source, a microwave emitter and any other electromagnetic energy emitter to cooperate with the bone cement. In FIGS. 4 and 5, the controller 155B can control all parameters of (i) heating the bone cement, (ii) the cement injection pressure and/or flow rate, (iii) energy delivery to cement flows in or proximate the distal end of the introducer and (iv) energy delivery to sense retrograde flows about the exterior surface of the introducer.

In one embodiment, as depicted in FIG. 5, the resistive heating element 210 can include a helically wound coil of a resistive material within the interior bore 122 of the introducer 120. The heating element 210 can optionally be further formed from, or coated with, a positive temperature coefficient material and coupled to a suitable voltage source to provide a constant temperature heater as is known in the art. As can be seen in FIG. 5, the heating element 210 can be carried within an insulative coating 232 in the interior of core sleeve 130, which is a conductive metal as described above.

FIG. 5 shows another aspect of certain embodiments, where it can be seen that the exterior surface of sleeve 120 has an insulative, scratch-resistant coating 132 that can include a thin layer of an insulative amorphous diamond-like carbon (DLC) or a diamond-like nanocomposite (DCN). It has been found that such coatings have high scratch resistance, as well as lubricious and non-stick characteristics that are useful in bone cement injectors, such as the injectors disclosed herein. Such coatings are particularly useful for an introducer sleeve 120 that carries electrical current for (i) impedance sensing purposes; (ii) for energy delivery to bone fill material; and/or (iii) ohmic heating of tissue. For example, when inserting a bone cement injector through the cortical bone surface of a pedicle and then into the interior of a vertebra, it is important that the exterior insulative coating portions do not fracture, chip or scratch to thereby insure that the electrical current carrying functions of the injector are not compromised.

The amorphous diamond-like carbon coatings and the diamond-like nanocomposites are available from Bekaert Progressive Composites Corporations, 2455 Ash Street, Vista, Calif. 92081 or its parent company or affiliates. Further information on the coating can be found at: http://www.bekaert.com/bac/Products/Diamond-like%20coatings.htm, the contents of which are incorporated herein by reference. The diamond-like coatings can be amorphous carbon-based coatings with high hardness and low coefficient of friction. The amorphous carbon coatings exhibit non-stick characteristics and excellent wear resistance. The coatings are thin, chemically inert and have a very low surface roughness. In one embodiment, the coatings have a thickness ranging between 0.001 mm and 0.010 mm; or between 0.002 mm and 0.005 mm. The diamond-like carbon coatings can be a composite of sp2 and sp3 bonded carbon atoms with a hydrogen concentration between 0 and 80%. Another diamond-like nanocomposite coatings (a-C:H/a-Si:O; DLN) is made by Bakaert and is suitable for use in the bone cement injectors disclosed herein. The materials and coatings are known by the names Dylyn®Plus, Dylyn®/DLC and Cavidur®.

FIG. 5 further illustrates another aspect of bone cement injector 105 (see FIG. 2) that again relates to the thermal energy emitter (resistive heater 210) within interior passageway 122 of introducer 120. In one embodiment, it has been found that it is advantageous to provide a lubricious surface layer 240 within the interior of resistive heater 210 to ensure uninterrupted cements flows through the thermal emitter without sticking. In one embodiment, surface layer 240 can be a fluorinated polymer such as Teflon or polytetrafluroethylene (PTFE). However, other suitable fluoropolymer resins can be used, such as FEP and PFA. Other materials also can be used, such as FEP (Fluorinated ethylenepropylene), ECTFE (Ethylenechlorotrifluoroethylene), ETFE (Ethylene tetrafluoroethylene), Polyethylene, Polyamide, PVDF (Polyvinylidene Difluoride), Polyvinyl chloride and silicone. The scope of the invention includes, but is not limited to, providing a bone cement injector having a flow channel extending therethrough with at least one open termination 125, wherein a surface layer 240 within the flow channel has a static coefficient of friction of less than 0.5, less than 0.2, or less than 0.1.

In another embodiment, the bone cement injector can have a flow channel 122 extending therethrough with at least one open termination 125, wherein at least a portion of the surface layer 240 of the flow channel is ultrahydrophobic or hydrophobic which may better inhibit a hydrophilic cement from sticking.

In another embodiment, the bone cement injector can have a flow channel 122 extending therethrough with at least one open termination 125, wherein at least a portion of the surface layer 240 of the flow channel is hydrophilic, which may inhibit a hydrophobic cement from sticking.

In another embodiment, the bone cement injector can have a flow channel 122 extending therethrough with at least one open termination in a distal end thereof, wherein the surface layer 240 of the flow channel has high dielectric strength, a low dissipation factor, and/or a high surface resistivity.

In another embodiment, the bone cement injector can have a flow channel 122 extending therethrough with at least one open termination 125 in a distal end thereof, wherein the surface layer 240 of the flow channel is oleophobic.

In another embodiment, the bone cement injector can have a flow channel 122 extending therethrough with at least one open termination 125 in a distal end thereof, wherein the surface layer 240 of the flow channel has a substantially low coefficient of friction polymer or ceramic.

In another embodiment, the bone cement injector can have a flow channel 122 extending therethrough with at least one open termination 125 in a distal end thereof, wherein the surface layer 240 of the flow channel has a wetting contact angle greater than 70°, greater than 85°, and greater than 100°.

In another embodiment, the bone cement injector can have a flow channel 122 extending therethrough with at least one open termination in a distal end thereof, wherein the surface layer 240 of the flow channel has an adhesive energy of less than 100 dynes/cm, less than 75 dynes/cm, and less than 50 dynes/cm.

The apparatus above also can be configured with any other form of thermal energy emitter that includes the non-stick and/or lubricious surface layer as described above. In one embodiment, the thermal energy emitter can comprise at least in part an electrically conductive polymeric layer. In one such embodiment, the electrically conductive polymeric layer has a positive temperature coefficient of resistance.

FIG. 6 illustrates one embodiment of a treatment device or system 380 that can be used for cutting or fracturing cancellous bone in a particular plane. The system 380 can include an elongated shaft 400 capable of a linear shape or configuration along a longitudinal axis, and a curved shape or configuration along at least a portion of the length of the shaft 400. As can be seen in FIG. 6, the shaft can be inserted through a thin-walled tubular sleeve 410, which can at least partially maintain the shaft in the linear configuration. The shaft 400 can be of a shape memory alloy. In one embodiment, the shaft 400 is preferably of a superelastic alloy such as Nitinol. In the embodiment of FIG. 6, it can further be seen that the elongated shaft 400 carries an elongated cutting element 420 that in one embodiment can be a wire-like member. The cutting element 420 is adapted to assume a first configuration co-linear the shaft 400, and adapted to assume a second configuration that in not co-linear with the shaft when the shaft 400 is curved for cutting cancellous bone 146 (see FIG. 3A).

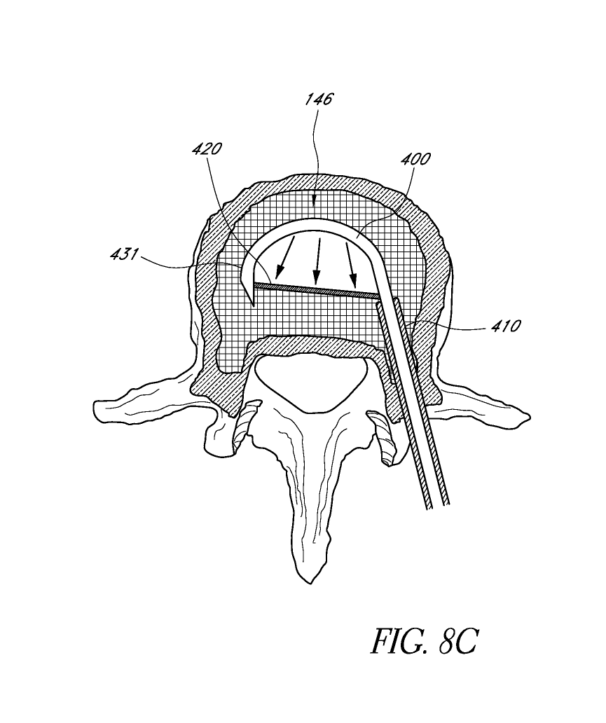

FIG. 7 shows the cutting element 420 carried in a channel 422 of the shaft 400 (see FIG. 6). The cutting element 420 can be maintained or constrained in the channel 422 (also see FIG. 6) by a snap fracturable material 425, or any rubber feature or snap fit features. However, other suitable mechanisms can be used to removably retain the cutting element 420 within the channel 422 of the shaft 400. When the working end of the device 380 is in the curved configuration and “locked” into cancellous bone 146 (as shown in FIG. 8B), a proximal end 428 (see FIGS. 8A-8C) of the cutting element 420 can be tensioned from the handle end (not shown) of the shaft 400 to cause the cutting element 420 to move from the first configuration co-linear with the shaft 400 to the second configuration not co-linear with the shaft 400 to thereby cut a planar path in the cancellous bone 146.

Thus, as seen in FIGS. 8A-8C, a method for creating a cavity in cancellous bone 146 can include providing the apparatus 380 described above, driving the shaft 400 distally from sleeve 410, positioning the shaft working end 431 in cancellous bone 146 in a curved shape, and actuating the cutting element 420 from a first configuration to a second configuration to thereby cut a plane in the cancellous bone 146 across the arc of the shaft working end 431. Such a method of treating bone is particularly directed to cancellous bone within a vertebra. The introduction of the shaft 400 can be through any cortical wall of a vertebra, and preferably through a pedicle. The method encompasses positioning the working end 431 by unconstraining a shape memory alloy shaft to provide the curved shape. In another embodiment, the apparatus and method utilize a pull wire to move the shaft 400 from the linear configuration to the curved configuration. In another embodiment (not shown) the shaft 400 also can have a bore therethrough, allowing the shaft 400 to be introduced into the vertebra over a guidewire. In still another embodiment, the shaft 400 also can have a rotatable drill tip.

In another embodiment, the shaft can have first and second cutting elements (not shown) on opposing sides of the shaft. After positioning the shaft within a bone (e.g., with contemporaneous imaging), a distally-oriented cutting element can be extended outwardly from the shaft with the contemporaneous application of low frequency vibration, ultrasound energy delivery, oscillation, rotation or axial movement to cut cancellous bone, or any other energy delivery method.

In general, the method encompasses an actuating step that includes applying energy from the cutting element to body structure. The energy-applying step can includes applying energy selected from a group of thermal energy, ultrasound energy, vibration energy, mechanical energy, light energy, electromagnetic energy, radiofrequency energy, microwave energy, chemical energy, and other forms of energy delivery. The effect of such energy delivery is for cutting tissue, coagulating tissue, sealing tissue, damaging tissue, vaporizing tissue, and other methods of tissue manipulation.

In a further method of treating a bone, an additional step includes introducing bone fill material into the cut plane. The bone fill material is preferably a flowable material such as an exothermic bone cement.

The method can include creating a cut plane that is adapted to control and direct the flow of bone cement to provide lesser height dimension and greater lateral dimension to the cement volume. The cement volume thus can be planar or a pancake-like distribution rather than a “round” bolus.

In another method, a shaft having an arc-configurable working end can be introduced into cancellous bone in a vertebra, the working end can be positioned to extend at least about 90° in an arc configuration within the cancellous bone, and the cutting element can be actuated across the arc configuration to thereby cut bone. The method further includes causing the working end to extend in an arc of at least about 120°, 150°, 180°, 210° and 240°.

In another method of treating a bone, complementary shafts with two working ends can be introduced, one from each pedicle. The shaft working end can overlap or can connect at distal portions thereof. In another embodiment and method, a cutting wire can be passed from one working end to the other to allow an abrasive wire to move axially from one instrument to the other by actuation from handles thereof. In any such embodiment, an energy source can be coupled to the cutting element.

In another embodiment and method, a flexible or shape memory bone cement injector, such as the injector 105 described above, can be introduced into the path created by the shaft 400, and then cement can be injected from a plurality of ports along the length of the injector working end, wherein the ports are oriented toward the cut plane. The working end of the injector can have the heating element as described above, or preferably a polymeric PTCR heating element. In such an embodiment, the step of applying thermal energy can be accomplished by a resistive heating element that has a sleeve fabricated of a positive temperature coefficient of resistance (PTCR) material.

In another embodiment, the step applying thermal energy can be accomplished by light energy from an LED, or from at least one of coherent light and non-coherent light.

In another embodiment, the step of applying thermal energy can include the heat of vaporization from a vapor, which can be introduced through a channel in the injector to interact with the cement. Such a vapor can be generated from water, saline or any other biocompatible fluid.

An injection system, such as those disclosed above, can use any suitable energy source, other that radiofrequency energy, to accomplish the purpose of altering the viscosity of the fill material 145. The method of altering fill material can be at least one of a radiofrequency source, a laser source, a microwave source, a magnetic source, an ultrasound source, or any other energy source. Each of these energy sources can be configured to preferentially deliver energy to a cooperating, energy sensitive filler component carried by the fill material. For example, such filler can be suitable chromophores for cooperating with a light source, ferromagnetic materials for cooperating with magnetic inductive heating means, or fluids that thermally respond to microwave energy.

The scope of the invention includes, but is not limited to, using additional filler materials such as porous scaffold elements and materials for allowing or accelerating bone ingrowth. In any embodiment, the filler material can comprise reticulated or porous elements of the types disclosed in co-pending U.S. patent application Ser. No. 11/146,891, filed Jun. 7, 2005, titled “Implants and Methods for Treating Bone” which is incorporated herein by reference in its entirety and should be considered a part of this specification. Such fillers also can carry bioactive agents. Additional fillers, or the conductive filler, also can include thermally insulative solid or hollow microspheres of a glass or other material for reducing heat transfer to bone from the exothermic reaction in a typical bone cement component.

The above description of the some embodiments of the invention is intended to be illustrative and not exhaustive. Particular characteristics, features, dimensions and the like that are presented in dependent claims can be combined and fall within the scope of the invention. The invention also encompasses embodiments as if dependent claims were alternatively written in a multiple dependent claim format with reference to other independent claims. Specific characteristics and features of the invention and its method are described in relation to some figures and not in others, and this is for convenience only. While the principles of the invention have been made clear in the descriptions and combinations, it will be obvious to those skilled in the art that modifications may be utilized in the practice of the embodiments of the invention, and otherwise, which are particularly adapted to specific environments and operative requirements without departing from the principles of the invention. The appended claims are intended to cover and embrace any and all such modifications, with the limits only of the true purview, spirit and scope of the invention.

Certain embodiments disclosed herein provide vertebroplasty systems and methods for sensing retrograde bone cement flows that can migrate along a fractured path toward a pedicle and risk leakage into the spinal canal. The physician can be alerted instantaneously of cement migration in a direction that can impinge on nerves or the spinal cord. Other embodiments include integrated sensing systems and energy delivery systems for applying energy to tissue and/or to bone cement that migrates in a retrograde direction wherein the energy polymerizes the cement and/or coagulates tissue to create a dam to prevent further cement migration. In another embodiment, the systems provide a cooling system for cooling bone cement in a remote container or injection cannula for controlling and extending the working time of a bone cement. In another embodiment, the bone cement injection system includes a thermal energy emitter for warming a chilled bone cement in the distal end of an injector or for applying sufficient energy to accelerate polymerization and thereby increase the viscosity of the bone cement.

A computer controller can be provided to control cement inflow parameters from a hydraulic source, the sensing system and energy delivery parameters for selectively heating tissue or polymerizing cement at both the interior and exterior of the injector to thereby control all parameters of cement injection to reduce workload on the physician.

In one embodiment, a lubricous surface layer is provided in the flow passageway of the bone cement injector to prevent sticking particularly when heating the cement.

Of course, the foregoing description is that of certain features, aspects and advantages of the certain embodiments of the present invention, to which various changes and modifications can be made without departing from the spirit and scope of the present invention. Moreover, the bone treatment systems and methods need not feature all of the objects, advantages, features and aspects discussed above. Thus, for example, those skilled in the art will recognize that the invention can be embodied or carried out in a manner that achieves or optimizes one advantage or a group of advantages as taught herein without necessarily achieving other objects or advantages as may be taught or suggested herein. In addition, while a number of variations of the invention have been shown and described in detail, other modifications and methods of use, which are within the scope of this invention, will be readily apparent to those of skill in the art based upon this disclosure. It is contemplated that various combinations or subcombinations of these specific features and aspects of embodiments may be made and still fall within the scope of the invention. Accordingly, it should be understood that various features and aspects of the disclosed embodiments can be combined with or substituted for one another in order to form varying modes of the discussed bone treatment systems and methods.

What is claimed is:

1. A device for treating bone, comprising:
an elongated shaft adapted for insertion through a cortical bone portion of a bone and into a cancellous bone portion of the bone, the elongated shaft having a linear shape extending along a longitudinal axis, at least a portion of the shaft being movable into a curved configuration within cancellous bone; and
a cutting element attached to said movable portion of the shaft, the cutting element moveable between a first configuration co-linear with the shaft and a second configuration away from the shaft with the movable portion of the shaft in the curved configuration to cut a plane in cancellous bone.

2. The device of claim 1, wherein the shaft comprises a shape memory alloy.

3. The device of claim 1, wherein the shaft comprises a superelastic alloy.

4. The device of claim 1, wherein the cutting element comprises a wire-like member.

5. The device of claim 1, wherein the cutting element is removably disposed within a channel defined by the shaft.

6. The device of claim 5, wherein the cutting element is removably attached to the channel by a snap fracturable material.

7. The device of claim 1, wherein the cutting element is coupleable to an energy source, the cutting element configured to apply energy to cancellous bone to cut cancellous bone.

8. The device of claim 7, wherein the energy source is chosen from a group consisting of: thermal energy, ultrasound energy, vibration energy, mechanical energy, light energy, electromagnetic energy, Rf energy, microwave energy and chemical energy.

9. A device for treating bone, comprising:
an elongated shaft adapted for insertion through a cortical bone portion of a bone and into a cancellous bone portion of the bone, the elongated shaft having a linear shape along a longitudinal axis, at least a portion of the shaft movable into a curved configuration within cancellous bone; and
means for cutting a plane in cancellous bone, said means being attached to at least a portion of the elongated shaft.

10. A method of treating bone, comprising:
creating a path into a cancellous bone portion in a bone;
inserting an elongated shaft along a longitudinal axis through said path into cancellous bone, the shaft having a cutting element attached to a working end of the shaft;
moving the working end of the shaft into a curved configuration; and
cutting a plane in cancellous bone with the cutting element.

11. The method of claim 10, wherein creating a path includes introducing the shaft through a pedicle.

12. The method of claim 10, wherein creating a path comprises inserting a sleeve in a minimally-invasive manner through an incision in a patient's skin.

13. The method of claim 10, wherein the inserting step includes contemporaneously imaging the insertion of the shaft.

14. The method of claim 10, wherein moving the working end of the shaft includes actuating a pull wire to provide the curved shape.

15. The method of claim 10, wherein cutting a plane includes at least one of rotating, oscillating and ultrasonically vibrating the cutting element.

16. The method of claim 10, wherein cutting a plane in cancellous bone comprises moving the cutting element from a first configuration co-linear the shaft to a second configuration away from the shaft.

17. The method of claim 16, wherein cutting a plane in cancellous bone further comprises applying energy to the cancellous bone via the cutting element.

18. The method of claim 17, wherein applying energy comprises applying energy chosen from a group consisting of: thermal energy, ultrasound energy, vibration energy, mechanical energy, light energy, electromagnetic energy, Rf energy, microwave energy and chemical energy.

19. The method of claim 10, further comprising flowing a bone cement into the plane in the cancellous bone to provide a planar volume of bone cement.

20. The method of claim 19, further comprising applying energy to the bone cement to alter viscosity thereof and polymerize the bone cement.

21. The method of claim 20, wherein applying energy comprises applying energy chosen from the group consisting of: electrical energy, thermal energy, RF energy, ultrasound energy, microwave energy and electromagnetic energy.

22. The method of claim 20, wherein applying energy further includes cutting tissue, coagulating tissue, sealing tissue, damaging tissue and vaporizing tissue.

23. The method of claim 10, wherein the bone is a vertebra.

24. A kit for treating a bone, comprising:
an injector configured for introduction into a bone, the injector configured to deliver a bone cement through a channel thereof into the bone; and
a cutting tool comprising an elongated shaft adapted for insertion through a cortical bone portion of the bone and into a cancellous bone portion of the bone, and a cutting element attached to said portion of the shaft, the cutting element moveable between a first configuration co-linear with the shaft and a second configuration not co-linear with the shaft to cut a plane in cancellous bone

25. The kit of claim 24, further comprising a sleeve insertable into the cancellous bone portion, the sleeve configured to receive the cutting tool therethrough into the cancellous bone portion.
